(19) United States
(12) Patent Application Publication
(10) Pub. No.: US 2022/0288369 A1
(43) Pub. Date: Sep. 15, 2022

(54) TARGETED DRUG DELIVERY DEVICES AND METHODS

(71) Applicant: Rithim Biologics, Inc., Woodbine, MD (US)

(72) Inventors: John Kevin Donahue, Holliston, MA (US); Ronald Lee Anderson, Woodbine, MD (US)

(73) Assignee: Rithim Biologics, Inc., Woodbine, MD (US)

(21) Appl. No.: 17/638,586

(22) PCT Filed: Aug. 27, 2020
(86) PCT No.: PCT/US20/48075

§ 371 Date: Feb. 25, 2022

Related U.S. Application Data
(60) Provisional application No. 62/892,660, filed on Aug. 28, 2019.

(51) Int. Cl. A61M 37/00; A46B 9/00; A46D 1/00; A46B 11/00

(52) U.S. Cl. CPC A61M 37/00; A46B 9/005; A46D 1/0253; A46B 11/0037; A46B 2200/10; A61M 2210/125

(57) ABSTRACT

A targeted drug delivery device comprises a brush for use in delivery of one or more therapeutic agents during surgical procedures, including but not limited to, heart, chest, abdominal, brain, limb, vascular, eye, organ transplantation surgery or cancer surgical treatment. The therapeutic agent is selected from the group consisting of genetically engineered vectors or cells, proteins, stem cells, small molecule pharmaceuticals, drugs or biologics. Methods of treating diseases comprising delivering at least one therapeutic agent to a patient's organs or tissues using any one of the targeted drug delivery devices are disclosed.

### CROSS REFERENCE

This application is a § 371 National Stage Application of PCT/US20/48075, filed Aug. 27, 2020, which claims the benefit of U.S. Provisional Application No. 62/892,660, filed Aug. 28, 2019, the entirety of which is hereby incorporated by reference.

### FIELD

This invention relates to drug delivery and therapy. More particularly, the present invention provides methods and apparatus for the delivery of one or more therapeutic agents onto or within the patient's target organ(s) such as heart, lung, blood vessel, intestine, pancreas, liver, kidney, brain or nerve, among others.

### BACKGROUND ART

Cardiac diseases are the most common causes of morbidity and mortality in the developed world. Coronary artery disease causing angina and myocardial infarction, cardiac arrhythmias causing stroke, syncope and sudden death, and heart failure causing disability and death are examples of cardiac diseases that greatly affect the lives of the afflicted as well as their families, employers and friends.

Limitations to prevention and therapy of cardiac diseases include the inability to directly target underlying disease mechanism for many of these diseases, and the limitations of off-target effects of systemically administered drugs that might otherwise be effective for other diseases. Potential clinical approaches to these limitations have included pharmacotherapy, gene therapy, cell therapy, protein and other biological therapies. To enhance efficacy and to limit off-target effects, it is desirable for these potential therapies to be delivered locally to the target portion(s) of the heart. In particular, there is a need for a device that can deliver therapeutic agent(s), such as genetically engineered vectors or cells, proteins, stem cells, small molecule pharmaceuticals, drugs or biologics, precisely to a target on or within a patient's heart.

Similarly, diseases of the lungs, blood vessels, nerves or other organs can be treated or prevented with locally delivered therapeutics during surgical procedures. These diseases include but are not limited to intestinal motility disorders, gastric motility disorders, diabetes mellitus, blood clotting disorders, metabolic diseases and renal failure among others. A need exists for methods and devices for local delivery of therapeutic agent(s) to the target organ. A system which delivers a controlled amount of an agent to the target organ, without creating additional tissue damage or significant inflammatory responses, would satisfy a great need in the art.

### SUMMARY OF THE INVENTION

In view of the foregoing, it is an object of the present invention to provide methods and apparatus for delivery of a therapeutic agent to the target organ or tissue to enhance therapeutic efficacy and to limit off-target effects.

The disclosed embodiments include a device that comprises a brush for use in delivery of one or more therapeutic agents during surgical procedures, including but not limited to, heart, chest, abdominal, brain, limb, vascular, eye, organ transplantation surgery or cancer surgical treatment.

One exemplary embodiment of the invention provides a device for delivering at least one therapeutic agent to an organ or tissue comprising a straight handle; and a brush consisting of a plurality of strands or sheets of a biocompatible or bioabsorbable material affixed to a distal end of the handle. Another embodiment of the invention provides a device for delivering at least one therapeutic agent comprising a straight handle; a fixed angle canted arm affixed to a distal end of the handle; a brush consisting of a plurality of strands or sheets of a biocompatible or bioabsorbable material affixed to a distal end of the canted arm. Alternatively, the handle and the canted arm could be integrally formed to be one piece of the canted handle. The strands may be looping strands attached to the handle in a continuous or interrupted fashion. The strands may be grooved to improve retention of the therapeutic agent(s) on the strand. Each strand may be monofilament or braided. The sheets may be stacked or wafered in equal lengths or varying lengths. Each sheet may have perforations configured to improve solution retention on the brush or may be solid without perforations.

Another exemplary embodiment of the invention provides a device for delivering at least one therapeutic agent to an organ or tissue comprising: a handle; a brush head affixed to a distal end of the handle; and a brush consisting of a plurality of strands or sheets of a biocompatible or bioabsorbable material affixed to the brush head. A further exemplary embodiment of the invention provides a device for delivering at least one therapeutic agent to an organ or tissue comprising: a handle; a fixed angle canted arm affixed to a distal end of the handle; a brush head affixed to a distal end of the canted arm; and a brush consisting of a plurality of strands or sheets of a biocompatible or bioabsorbable material affixed to the brush head. The handle and the brush head may be made of medical grade plastic. The brush head comprises an optional open slit for delivery of the therapeutic agent onto the brush. The brush head further comprises an external bar configured for affixing strands or sheets to the brush head. The strands or sheets of a biocompatible or bioabsorbable material are affixed to the brush head using the external bar with each strand or sheet being tied or glued to the bar, or crimped between the bar and the brush head. The strands may be looping strands attached to the brush head in a continuous or interrupted fashion. The strands may be grooved to improve retention of the therapeutic agent(s) on the strand. Each strand may be monofilament or braided. The sheets may be stacked or wafered in equal lengths or varying lengths. Each sheet may have perforations configured to improve solution retention on the brush or may be solid without perforations.

Still another exemplary embodiment of the invention provides a device for delivering at least one therapeutic agent to an organ or tissue comprising: a hollow handle having a central lumen for delivering the therapeutic agent(s) and an exterior tubular wall with at least one channel inside the wall for a control cable to pass through; a luer lock connector attached to a proximal end of the handle; a brush deflector control mounted on the handle configured to deflect a brush head at various angles; an articulating arm flexibly connected to a distal end of the handle; a brush head affixed to a distal end of the articulating arm; and a brush consisting of a plurality of strands or sheets of a biocompatible or bioabsorbable material affixed to the brush head. The central lumen allows a flexible inner tube or catheter to pass through for delivering the therapeutic agent to the brush. The luer lock connector may be connected to a flexible external tube or catheter to allow the therapeutic agent to flow from the external tube or catheter, through the handle and onto the brush. The articulating arm comprises an articulating arm central lumen for delivering the therapeutic agent(s) and at least one articulating arm channel for the bidirectional control cable to pass through. The brush deflector control is configured to deflect the brush head at various angles through the control cable and a slide mechanism. The brush head comprises an open slit for delivery of the therapeutic agent(s) onto the brush. The brush head further comprises an external bar configured for affixing strands or sheets to the brush head. The strands or sheets of a biocompatible or bioabsorbable material are affixed to the brush head using the external bar with each strand being tied or glued to the bar, or crimped between the bar and the brush head. The strands may be looping strands attached to the brush head in a continuous or interrupted fashion. The strands may be grooved to improve retention of the therapeutic agent(s) on the strand. Each strand may be monofilament or braided. The sheets may be stacked or wafered in equal lengths or varying lengths. Each sheet may have perforations configured to improve solution retention on the brush or may be solid without perforations.

Still another exemplary embodiment of the invention provides a syringe-type device for delivering at least one therapeutic agent to an organ or tissue comprising: a plunger handle; a threaded plunger shaft having a proximal end attached to the plunger handle; a plunger head connected to a distal end of the threaded plunger shaft; a syringe barrel having internal threads and having an opening at a proximal end thereof defined by an upper edge; a side port located on the side of the syringe barrel with a stopcock connection or a self-sealing valve for introduction of additional solution or therapeutic agent(s); an insulating layer coating a surface of the syringe barrel; a removable insulating sleeve encompassing the syringe barrel to maintain therapeutic temperature or allow comfortable handling; a one-way stopcock connected to a distal end of the syringe barrel; a brush head connected to a distal end of the stopcock; and a brush consisting of a plurality of strands or sheets of a biocompatible or bioabsorbable material affixed to the brush head; wherein the plunger shaft comprises external threads to engage the internal threads of the syringe barrel for advancing or retracting the plunger shaft by rotating the plunger handle in a clockwise or counter clockwise direction. The one-way stopcock allows air ventilation when additional material is introduced through the side port. The one-way stopcock is closed when mixing material within the syringe and is opened when introducing material/solution into the brush head and onto the brush.

Any type of the plunger handle of the syringe-type device may be employed as should be apparent to those skilled in the art for powered or manual advancement. In one embodiment of the invention, the plunger handle of the syringe-type device comprises a disk-like or coin-like vertical member permanently affixed on top of another disk-like or coin-like horizontal base member. In another embodiment, the plunger handle comprises a flat disk-like or coin-like member with holes at a top surface of the member. Still in another embodiment, the plunger handle comprises a flat disk-like or coin-like member optionally having ridges at the edge of the member for manual advancement.

The brush head of the syringe-type device comprises a closed-end tube with a longitudinal slit opening and an external bar configured for affixing strands or sheets to the brush head. The strands or sheets of a biocompatible or bioabsorbable material are affixed to the brush head using the external bar with each strand or sheet being tied or glued to the bar, or crimped between the bar and the brush head. The strands may be looping strands attached to the brush head in a continuous or interrupted fashion. The strands may be grooved to improve retention of the therapeutic agent(s) on the strand. Each strand may be monofilament or braided. The sheets may be stacked or wafered in equal lengths or varying lengths. Each sheet may have perforations configured to improve solution retention on the brush or may be solid without perforations.

The syringe barrel of the syringe-type device comprises a threaded female shaft access and an upper edge in a proximal end for locking a power driver onto the syringe barrel. The syringe power driver configured to drive the plunger shaft of the syringe-type device comprises: an exterior case; a battery/power source situated at a proximal end of the exterior case; a circuitry situated next to a distal end of the battery/power source to control the revolution speed of the plunger shaft, shaft movement direction; a switch located on an outside surface of the exterior case with on/off, variable speed, and forward/reverse controls of the rotation of the plunger shaft; a motor situated next to a distal end of the circuitry; a drive shaft situated in between the motor and a drive shaft extension and having a proximal and distal end connected to the motor and the drive shaft extension, respectively; the drive shaft extension; and a plurality of connector ridges at a distal end of the exterior case to secure the syringe power driver onto the syringe barrel by twist connection onto the upper edge of the syringe barrel; wherein the motor is configured to drive the drive shaft and the drive shaft extension forward or backward within the exterior case; and wherein the drive shaft along with the drive shaft extension is equal in length to the syringe plunger shaft when being fully retracted. The syringe power driver is configured to be used in a sterile or non-sterile surgical environment. The configuration of the components and structures situated within the exterior case may be varied to obtain desired operations without departing from the scope of the present invention.

The drive shaft extension of the syringe power driver may employ any type of the engagement mean as should be apparent to those skilled in the art. In one embodiment, the drive shaft extension comprises an elongated engagement slit configured to engage one type of a plunge handle by allowing the vertical member of the plunger handle to be inserted into the slit. Another embodiment of the drive shaft extension comprises a plurality of engagement pins configured to fit into the holes of another type of the syringe plunger handle with holes at the top surface of a flat disk-like or coin-like member. The configuration of the engagement pins of the drive shaft extension and the corresponding holes of the syringe plunger handle may be varied to obtain desired operations without departing from the scope of the present invention.

Still another exemplary embodiment of the invention provides a device for delivering at least one therapeutic agent to a posterior or distal surface of an organ or tissue comprising a platform handle having one or more finger controls mounted on a proximal end of the handle and a brush at a distal end of the handle, wherein the handle may be made of flexible medical grade plastic. Other materials may be employed in the alternative, as should be apparent to those skilled in the art. The brush comprises a biocompatible or bioabsorbable material in various configurations inclusive but not limited to single strands, multiple strands, looping, single or multiple sheets, solid or perforated, or wafered. The brush may cover one side of the device, and the opposite side of the device may comprise at least one finger control in the shape of rings, semi-circular, a single bar, or other structures conducive to holding controls so that an operator would place a finger or fingers through to support or guide the device during application of the therapeutic agent(s). Another embodiment of the platform handle may comprise a proximal port connected to a channel or tube within the platform terminating into an opening on the brush side of the platform and on the opposite side from the finger controls. The proximal port of the platform may have a luer lock or similar connector to facilitate connection of a tube or syringe to deliver the therapeutic agent(s) or solution through the channel and onto the brush. Yet another embodiment of the platform handle may provide a proximal port comprising a connector to facilitate connection of a flexible or squeezable reservoir to the channel or tube within the platform and terminating into an opening on the brush side of the platform and on the opposite side from the finger controls. The reservoir may have a port or luer lock for filling the reservoir with a therapy or solution.

The disclosed embodiments also include a method of treating diseases comprising delivering at least one therapeutic agent to a patient's organs or tissues using any one of the devices described herein. An embodiment involves a therapeutic agent selected from the group consisting of genetically engineered vectors or cells, proteins, stem cells, small molecule pharmaceuticals, drugs or biologics. The disclosed methods of treatment also pertain to treatment or prevention of cardiac diseases like conduction system disease, sinus node dysfunction, atrial fibrillation, myocardial infarction, and heart failure and prevention of stroke that may result from embolization of thrombus formed in the cardiac left atria or pulmonary embolism from thrombus formed in the right atrium.

Other embodiments include methods for treating other organ-based diseases by delivery of therapeutic nucleotides, genes, cells, proteins, drugs or biologics to relevant target organs, including the brain, nerve, pancreas, lungs, blood vessels, liver, kidneys, stomach or intestines.

### BRIEF DESCRIPTION OF THE DRAWINGS

FIG. 1 shows the basic version of the device, comprising a straight handle affixed to the brush of a biocompatible or bioabsorbable material.

FIG. 2 shows the basic version of the device, comprising a slightly canted handle affixed to the brush of a biocompatible or bioabsorbable material.

FIG. 3 shows the basic version of the device, comprising a straight, solid handle affixed to a brush head.

FIG. 4 shows the device as constructed in FIG. 3 but modified to include a fixed angled canted arm.

FIG. 5 shows a modification of the device in FIG. 4 where the brush head can be deflected at various angles controlled by a brush deflector control mounted on a handle through a slide mechanism.

FIGS. 6a-6c show exemplary embodiments of a syringe-type handle with a screw plunger for manual advancement or for engagement of a power driver. FIG. 6a shows the first embodiment of the plunger handle; FIG. 6b shows the second embodiment of the plunger handle;

FIG. 6c shows the third embodiment of the plunger handle.

FIGS. 7a-7b show a syringe power driver and drive shaft extension. FIG. 7a shows a syringe power driver with a drive shaft extension having a syringe handle engagement slit. FIG. 7b shows a drive shaft extension having syringe handle engagement pins.

FIGS. 8a-8f show different brush types. FIG. 8a shows a brush comprising multiple individual looping strands of a biocompatible or bioabsorbable material mounted on the brush head mount in either a “running” fashion or “interrupted” fashion. FIG. 8b shown a brush comprising multiple individual single strands of a biocompatible or bioabsorbable material mounted on the brush head mount. FIG. 8c shows a brush comprising multiple layered sheets of a biocompatible or bioabsorbable material mounted on the brush head mount. Each sheet has perforations to improve solution retention on the brush. FIG. 8d shows a brush comprising a single sheet of a biocompatible or bioabsorbable material mounted on the brush head mount. The sheet has perforations to improve solution retention on the brush. FIG. 8e shows a brush comprising multiple layered solid sheets of a biocompatible or bioabsorbable material mounted on the brush head mount. FIG. 8f shows a brush comprising a single solid sheet of a biocompatible or bioabsorbable material mounted on the brush head mount.

FIG. 9 shows a specialized configuration of strands of a biocompatible or bioabsorbable material whereby grooves are created circumferentially in a strand.

FIGS. 10a-10c show exemplary embodiments of a brush handle with two finger controls for posterior epicardial therapeutic agent delivery. FIG. 10a shows the first embodiment of the brush handle; FIG. 10b shows the second embodiment of the brush handle; FIG. 10c shows the third embodiment of the brush handle.

### DETAILED DESCRIPTION OF THE EMBODIMENTS

All patents and other publications identified are incorporated herein by reference for the purpose of describing and disclosing, for example, the methodologies described in such publications that might be used in connection with the present invention, but are not to provide definitions of terms inconsistent with those presented herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on information available to the applicants and do not constitute any admission as to the correctness of the dates or contents of these documents.

As used herein and in the claims, the singular forms “a,” “an,” and “the” include the plural reference unless the context clearly indicates otherwise. Throughout this specification, unless otherwise indicated, “comprise,” “comprises” and “comprising” are used inclusively rather than exclusively, so that a stated integer or group of integers may include one or more other non-stated integers or groups of integers. The term “or” is inclusive unless modified, for example, by “either.” Other than in the operating examples, or where expressly stated or otherwise indicated, all numbers expressing quantities of ingredients or reaction conditions used herein should be understood as modified in all instances by the term “about.”

The term “biocompatible” refers to the ability of a material to perform with an appropriate host response in a specific situation. The term “bioabsorbable” refers to the ability of being assimilated into the body.

As used herein, “fluid” refers primarily to liquids, but can also include suspensions of solids dispersed in liquids (dispersions, suspensions, colloidal mixtures), colloidal mixtures, emulsions, liposomal compositions, pluronic mixtures, and gasses dissolved in or otherwise present together within liquids inside the fluid-containing portions of syringes.

A “dosage form” or “formulation” refers to a drug or drug product which includes the active agent and may further include inactive substances such as excipients or diluents as are known in the art. The active agent may be a biologic, such as an antibody, protein, peptide or nucleic acid. A container used in conjunction with the drug delivery devices described herein, configured to deliver a selected dose, may include an additional volume of dosage form to account for “loss” in the delivery device.

References to “pharmaceutical agent,” “pharmaceutically active,” “pharmaceutical,” “drug,” “medicament,” “active agent,” “active drug” “active pharmaceutical ingredient,” “API,” and the like, refer in a general sense to substances useful in the medical and scientific arts as suitable for delivery via a syringe, including, for example, drugs, biologics, diagnostic agent(s) (e.g., dyes or contrast agent(s)) or other substances used for therapeutic, diagnostic, or preventative (e.g., vaccines), or research purposes. Example pharmaceutical agent(s) include biologics, vaccines, chemotherapeutic agent(s), contrast agent(s), small molecules, immunogens, antigens, interferons, polyclonal antibody preparations, monoclonal antibodies, anesthetics, interfering RNAs, gene vectors, insulins, or combinations of any of these. As noted, a dosage form may comprise one or more active therapeutic agent(s), or a combination of active and diagnostic agent(s), etc.

“Inactive” substances refer to carriers, excipients, diluents, and the like, which are well-known in the art, although such substances may have beneficial function in the mixture, such as, for example, pluronic, surfactant, inorganic or organic salt, stabilizer, diluent, solubilizer, reducing agent, antioxidant, chelating agent, preservative, adjuvants, isotonic or buffering agent(s), or any excipient conventionally used in pharmaceutical compositions (i.e., “pharmaceutically acceptable excipient”) and the like. These active or inactive substances may also include substances having immediate, delayed, controlled, or sustained release characteristics.

A “dosage form,” “pharmaceutical formulation,” “formulation,” or “pharmaceutical composition” refers to a drug product that includes at least one active agent and may further include pharmaceutically acceptable excipients, carriers, buffers, stabilizers, or other materials well known to those skilled in the art. For example, a typical injectable pharmaceutical formulation includes a parenterally acceptable aqueous solution which is pyrogen-free and has suitable pH, isotonicity, and stability. The dosage forms delivered by the devices disclosed herein can have diagnostic, therapeutic, cosmetic, or research utility in various species, such as for example in human patients or subjects.

The term “therapeutic agent” as used herein refers to any therapeutically active substance that is administered to a subject to produce a desired, usually beneficial, effect. The term therapeutic agent includes, e.g., classical low molecular weight therapeutic agent(s) commonly referred to as small molecule drugs; and biologics including, but not limited to, antibodies or functionally active portions thereof, peptides, lipids, protein drugs, protein conjugate drugs, fusion proteins, enzymes, nucleic acids, ribozymes, genetic material, viruses, bacteria, eukaryotic cells, and vaccines. A therapeutic agent can also be a pro-drug, which is metabolized into the desired therapeutically active substance at or after administration to a subject. In some aspects, the therapeutic agent is a prophylactic agent. In addition, the therapeutic agent can be pharmaceutically formulated. A therapeutic agent can also be a radioactive isotope. A therapeutic agent can be an agent activated by a form of energy such as light or ultrasonic energy, or activated by other circulating molecules that can be administered systemically or locally.

A pharmaceutical formulation can include a therapeutically effective amount of at least one active agent. Such effective amounts can be readily determined by one of ordinary skill in the art based, in part, on the effect of the administered dosage form, or the combinatorial effect of an agent and one or more additional active agent(s), if more than one agent is used. A therapeutically effective amount of an active agent can also vary according to factors such as the disease state, age, sex, and weight of the individual, and the ability of the agent (and one or more additional active agent(s)) to elicit a desired response in the individual, e.g., amelioration of at least one condition parameter. For example, a therapeutically effective amount of a dosage form can inhibit (lessen the severity of or eliminate the occurrence of), prevent a particular disorder, or lessen any one of the symptoms of a particular disorder known in the art or described herein. A therapeutically effective amount may also be one in which any toxic or detrimental effects of the active agent or dosage form are outweighed by the therapeutically beneficial effects.

Accordingly, an active agent can be administered to a subject as a monotherapy. Alternatively, an active agent can be administered to a subject as a combination therapy with another active agent in a combination dosage form, or as an additional treatment, e.g., another treatment for an associated or additional disorder. For example, combination therapy can include administering to the subject (e.g., a human patient) one or more agent(s) (e.g., antibiotics, anti-coagulants, anti-hypertensives, or anti-inflammatory drugs) that provide a therapeutic benefit to a subject. In some embodiments, an active agent and one or more additional active agent(s) are administered in a single dosage form. In other embodiments, an active agent is administered first in time and an additional active agent(s) is administered second in time. In some embodiments, one or more additional active agent(s) are administered at the same time, but using different drug delivery devices or delivery modes.

A dosage form delivered according to the devices described herein may replace or augment a previously or currently administered therapy. For example, upon treating with one pharmaceutical formulation, administration of an additional active agent(s) can cease or be diminished, e.g., be administered at lower concentrations or with longer intervals between administrations. In some embodiments, administration of a previous therapy can be maintained. In some embodiments, a previous therapy is maintained until the level of an active agent reaches a level sufficient to provide a therapeutic effect. Accordingly, two therapies can be administered in combination, sequentially, or simultaneously.

The term “antibody” includes a full antibody; a derivative, portion, or fragment thereof, such as a fragment derived from enzymatic or chemical cleavage or a portion obtained recombinantly; or a mimic of the binding region of an antibody produced either by way of protein expression techniques or through chemical synthesis, which retains functionality as a specific binding member, such as the specific binding activity of at least one antibody antigen-binding domain site. Accordingly, the term antibody includes monoclonal antibodies and all the various forms derived from antibodies, including but not limited to full-length antibodies (e.g., having an intact Fc region), bifunctional antibodies, trifunctional antibodies, antigen-binding fragments (e.g., produced via enzymatic cleavage) or portions (e.g., polypeptides produced using recombinant methods) including, for example, scFv, di-scFv, sdAb, BiTE (bi-specific T-cell engager), Fab, Fab′ and F(ab′)2 fragments, diabodies, single chain antibodies, and other specific binding members comprising an antibody antigen-binding domain site. The terms “antibody” and “antibodies” as used herein also refer to human antibodies produced for example in transgenic animals or through phage display, as well as chimeric antibodies, humanized antibodies, and fully humanized antibodies or portions thereof that function as a specific binding member.

Biologics that can be advantageously delivered by the drug delivery devices as described herein include dosage forms comprising polymer solvent gels such as Kollipher® (poloxamer F127), Eligard® (leuprolide acetate for injectable suspension); dosage forms comprising polymer solutions such as gelatin, hyaluronic acid (Hyalgan®), hylan GF 20 (Synvisc-One®), or a mixture of cyclodextrin and polymeric hyaluronate or polymeric hyaluronic acid (see U.S. Pat. No. 9,089,478) or cross-linked hyaluronic acid (U.S. Pat. No. 9,050,336), fibrin/thrombin mixtures and other blood clotting agent(s); dosage forms comprising oily formulations, such as fulvestrant (Faslodex®); dosage forms comprising flowable polymer formulations, see WO2002030393 for example polymer microspheres, such as Lupron Depot® (leuprolide acetate for depot suspension); dosage forms comprising biologics, such as cells, platelets, cellular extracts, hormones, lubricin (proteoglycan), cytokines (e.g., granulocyte colony-stimulating factor), biomolecules having either agonist or antagonist activity (e.g., ligands or receptors), fusion proteins (such as a macromolecule having at least first and second functional moieties). References to biologics include variants, analogs, or derivatives thereof, such as pegylated filgastrim.

Brush material could consist of synthetic bioabsorbable materials such as polydioxanone (PDS); polylactic acid (PLA); polyglycolic acid (PGA); Polycaprolactone (PCL): or polylactic-co-glycolic acid (PLGA).

The term “absorbable” was recognized by IUPAC as the process of penetration and diffusion of a substance into another substance as a result of the action of attractive phenomena. In the context of biomaterials, “absorbable” refers to the uptake of substances into or across human or animal tissue. “Absorbable” is the best grammatical modifier with respect to our claims. Other descriptors include: bioabsorbable, resorbable, bioresorbable, degradable, biodegradable, and potentially other similar themes.

Methods of securing the brush strands or sheets to the brush head mount could be but not limited to: crimping; gluing with a medical grade adhesive; fixing or tying strands or sheets to perforations in the brush head mount or to a staple in the brush head mount; or any acceptable manufacturing process accepted for medical devices.

Non-limiting examples of cardiac diseases that can be treated using the devices described herein include conduction system disease, sinus node dysfunction, atrial fibrillation, myocardial infarction, heart failure, and inherited arrhythmia syndromes (including but not limited to long QT syndrome, Brugada Syndrome, Catecholaminergic Polymorphic Ventricular Tachycardia, Right Ventricular Cardiomyopathy/Dysplasia, early repolarization syndrome, and short QT syndrome). Other cardiac diseases may include rheumatic heart disease, hypertensive heart disease, ischemic heart disease, cerebrovascular disease, inflammatory heart disease, avalvular heart disease, aneurysm, atherosclerosis, peripheral arterial disease, angina, coronary heart disease, heart attack, stroke, transient ischemic attacks, cardiomyopathy, pericardial disease, valvular heart disease, congenital heart disease, cardiac arrhythmia, cardiomegaly, and heart neoplasia. Additional cardiac diseases may include acute decompensated heart failure, arteriosclerotic heart disease, athletic heart syndrome, atrioventricular fistula, autoimmune heart disease, Brown atrophy of the heart, Ebb Cade, cardiac amyloidosis, cardiac asthma, cardiac contractility modulation, cardiac syndrome X, cardiogenic shock, cardiophobia, cardiorenal syndrome, cardiotoxicity, carditis, coital angina, coronary artery aneurysm, coronary artery anomaly, coronary artery disease, coronary artery dissection, coronary artery ectasia, coronary occlusion, coronary steal, coronary thrombosis, coronary vasospasm, Coeur en sabot, Coxsackievirus-induced cardiomyopathy, diastolic heart failure, Dressler syndrome, Duroziez's disease, Eisenmenger's syndrome, embryocardia, embryonic recall, endocardial fibroelastosis, heart failure with preserved ejection fraction, high-output heart failure, hyperdynamic precordium, idiopathic giant-cell myocarditis, interventricular dyssynchrony, intraventricular dyssynchrony, isolated atrial amyloidosis, Keshan disease, Kounis syndrome, myocardial bridge, myocardial disarray, myocardial rupture. myocardial scarring. myocardial stunning, myocarditis, nonbacterial thrombotic endocarditis, Ostial disease, phosphorus and non-atherosclerotic heart disease, postpericardiotomy syndrome, pressure-controlled intermittent coronary sinus occlusion (PICSO), right axis deviation, Roemheld syndrome, Shone's syndrome, subacute bacterial endocarditis, traumatic cardiac arrest, ventricular aneurysm, and viral cardiomyopathy.

Non-limiting examples of lung diseases that can be treated using the devices described herein include cystic fibrosis (CF) lung disease, α-1-antitrypsin (AAT) deficiency lung disease, lung cancer, malignant mesothelioma, pulmonary inflammation, surfactant deficiency, and pulmonary hypertension.

Non-limiting examples of gastrointestinal diseases that can be treated using the devices described herein include inflammatory bowel disease, celiac disease, Crohn's disease, colon cancer,

Non-limiting examples of liver diseases that can be treated using the devices described herein include acute intermittent porphyria, chronic viral hepatitis, haemophilia A, haemophilia B, homozygous familial hypercholesterolaemia, al-antitrypsin, mucopolysaccharidosis VI, ornithine transcarbamylase deficiency, urea cycle defects, organic acidurias, phenylketonuria, glycogen storage disease type Ia, long chain fatty acid oxidation disorders, homozygous familial hypercholesterolemia, primary hyperoxaluria type I and progressive familial intrahepatic cholestasis.

Non-limiting examples of pancreatic diseases that can be treated using the devices described herein include pancreatitis, pancreatic cancer, cystic diseases, pancreatic divisum, islet cell tumors, endocrine tumors, diabetes mellitus, and pancreatic pain.

Non-limiting examples of kidney diseases that can be treated using the devices described herein include renal cell carcinoma, alport syndrome, glomerulonephritis and renal fibrosis.

Unless defined otherwise, all technical and scientific terms used herein have the same meaning as those commonly understood to one of ordinary skill in the art. The terminology used herein is for the purpose of describing particular embodiments only, and is not intended to limit the scope of the present invention, which is defined solely by the claims. The terms male and female may be used interchangeably to describe corresponding components or complementary aspects thereof and are not a limitation to either particular member unless context clearly indicates otherwise.

Headings are provided for convenience only and are not to be construed to limit the invention in any way. Unless defined otherwise, all technical and scientific terms used herein have the same meaning as those commonly understood to one of ordinary skill in the art. The terminology used herein is for the purpose of describing particular embodiments only, and is not intended to limit the scope of the present invention, which is defined solely by the claims. In order that the present disclosure can be more readily understood, certain terms are first defined. Additional definitions are set forth throughout the detailed description.

The disclosed embodiments include a brush system to deliver the desired therapeutic material(s) to the whole heart, to the whole of individual chambers of the heart (left atrium, right atrium, left ventricle, right ventricle), or to specific areas within the chambers (including but not limited to the coronary arteries, a single coronary artery or portion thereof, the sinus node, the right ventricular outflow track, a myocardial infarction scar or the border zone around a myocardial infarction scar), a blood vessel or multiple blood vessels, the lungs, kidneys, small or large intestines, stomach, liver, pancreas, with targeting of a particular organ in whole or in part. The brush could target a single area or multiple areas or the whole of the target organ. Delivered therapeutics could include individually or in combination small molecules, cells, gene(s), gene transfer vectors (including but not limited to viral vectors, non-viral vectors, or manufactured particles), proteins, lipids, carbohydrates, antibodies, or polymers, or any therapeutic substance or solution. The therapeutic could be delivered as a liquid if it exists in that state, or it could be delivered in a solution composed of a biocompatible substance, including but not limited to normal saline, phosphate buffered saline, water, ethanol, DMSO, or electrolyte solutions (e.g., lactated ringers solution). Additionally, to assist in keeping the therapeutic agent at the target site, it may be administered attached to a targeting agent such as an antibody or protein, or it may be administered in a gel or semi-solid form associated with a polymer (e.g., pluronics, matrigel, polylysine, collagen), carbohydrate, lipid emulsion or other substance that would allow the therapeutic agent to stick to its target. Additionally the polymer could allow immediate release of the therapeutic agent or it could have controlled release properties.

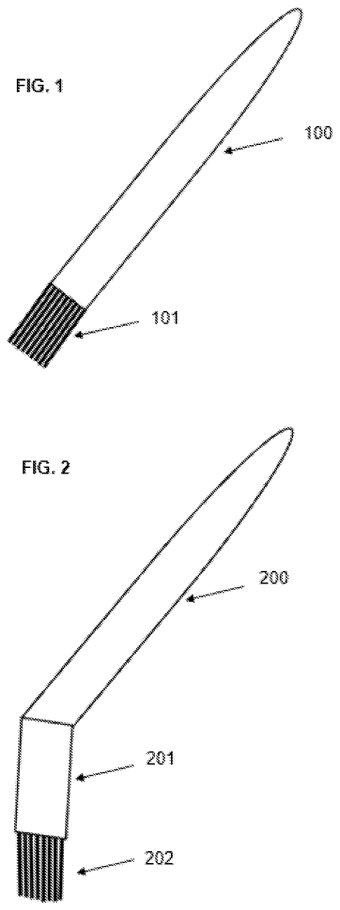

Several illustrative embodiments of a device suitable for use in conjunction with the present invention are now described below and depicted in FIGS. 1-10.

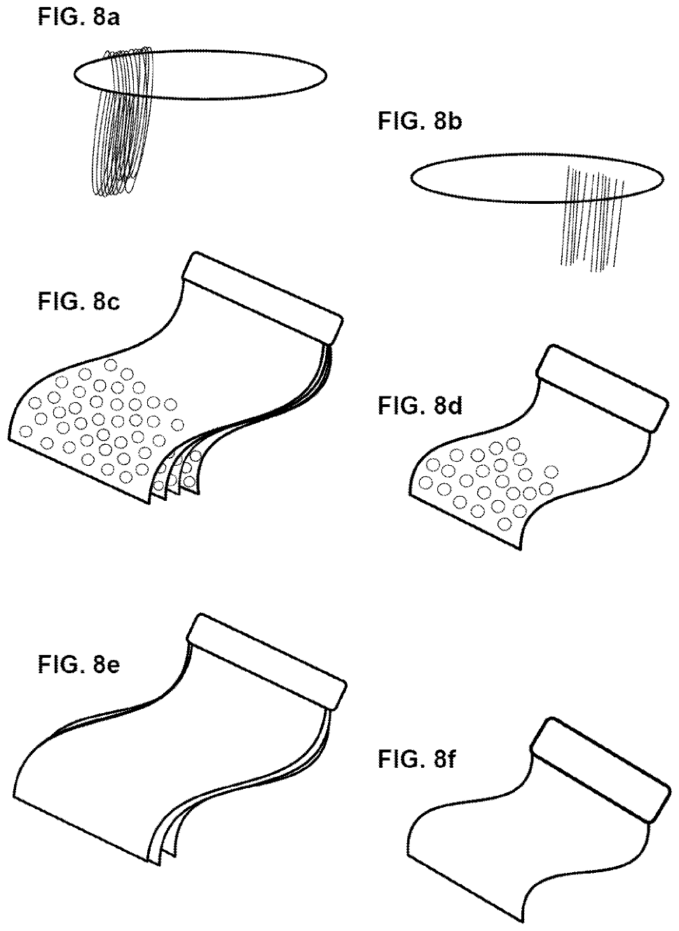

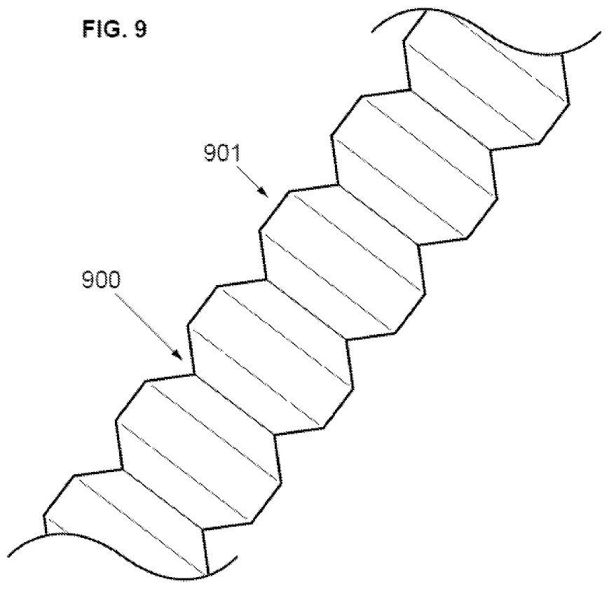

FIG. 1 shows a device for use in delivery of the therapeutic agent(s) according to one embodiment of the invention which comprises a straight handle 100 having a distal end affixed to a plurality of strands or sheets of a biocompatible or bioabsorbable material forming a brush 101. The brush may be of various configurations as shown in FIGS. 8a-8f.

FIG. 2 depicts a device for use in delivery of therapeutic agent(s) according to a second embodiment of the invention which comprises a straight handle 200 having a distal end connected to a canted arm 201. Alternatively, the handle 200 and the canted arm 201 may be integrally formed to be one piece of the canted handle. The canted arm 201 is affixed to a plurality of strands or sheets of a biocompatible or bioabsorbable material forming a brush 202. The brush may be of various configurations as shown in FIGS. 8a-8f.

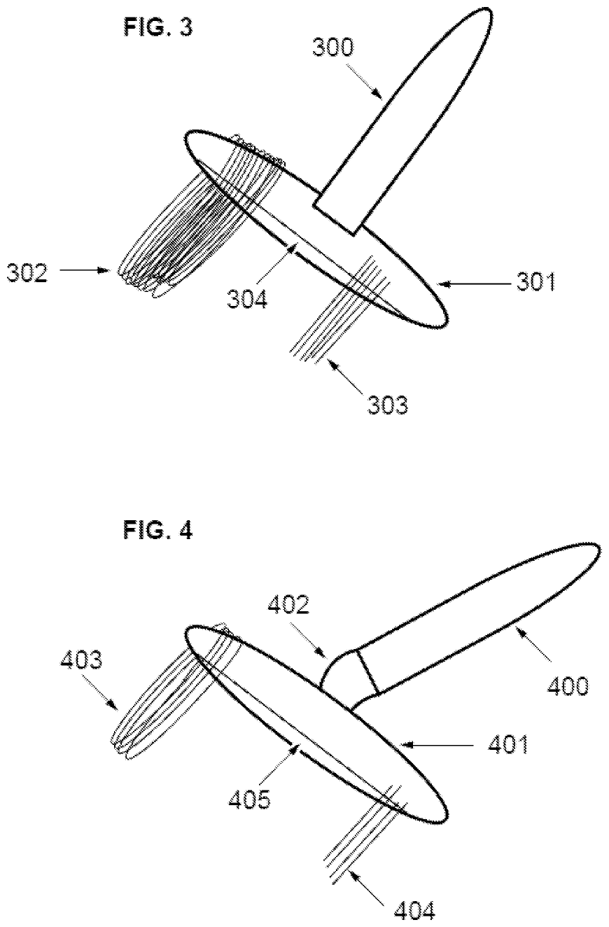

FIG. 3 shows a device for use in delivery of therapeutic agent(s) according to a third embodiment of the invention which comprises a straight, solid handle 300 having a distal end affixed to a brush head 301 at an angle, such as a 90-degree angle. The brush head comprises an optional open slit 304 for delivering a therapeutic agent onto a plurality of strands or sheets of a biocompatible or bioabsorbable material forming a brush. In another embodiment where the open slit 304 is absent, the brush head may be pre-loaded with the therapeutic agent(s), for example, by dipping into a container holding the therapeutic agent(s). The strands may be looping strands 302 attached to the brush head in a continuous or interrupted fashion. The strands may also be single straight strands 303. The straight strands may be of a single length or varying lengths. The brush may be of various configurations as shown in FIGS. 8a-8f.

FIG. 4 shows a device for use in delivery of therapeutic agent(s) according to a fourth embodiment of the invention which comprises a handle 400 having a distal end affixed to a brush head 401 at a fixed angle through a canted arm 402. The brush head comprises an optional open slit 405 for delivering a therapeutic agent onto a plurality of strands or sheets of a biocompatible or bioabsorbable material forming a brush. In another embodiment where the open slit 405 is absent, the brush head may be pre-loaded with the therapeutic agent(s), for example, by dipping into a container holding the therapeutic agent(s). The strands may be looping strands 403 attached to the brush head in a continuous or interrupted fashion. The strands may also be single straight strands 404. The brush may be of various configurations as shown in FIGS. 8a-8f.

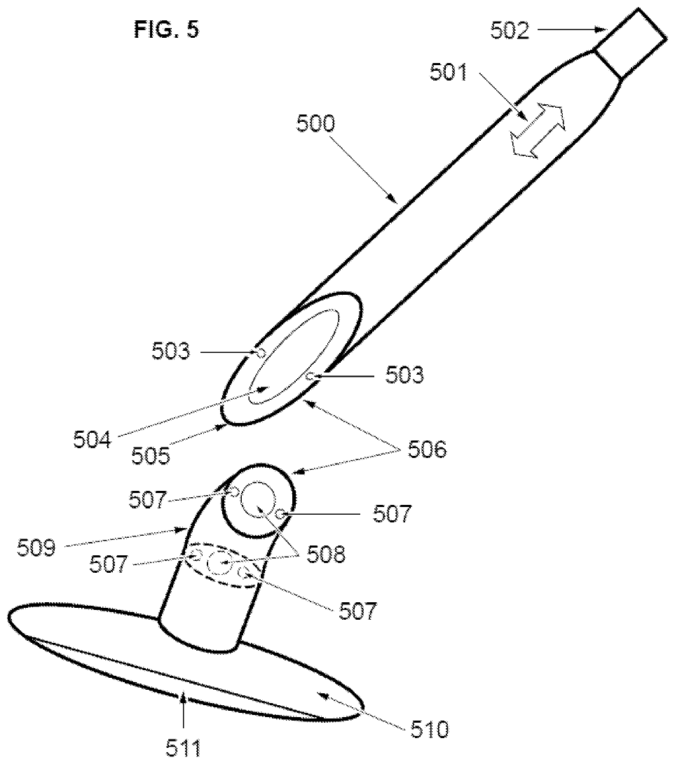

FIG. 5 shows a device for use in delivery of therapeutic agent(s) according to a fifth embodiment of the invention which comprises a hollow handle 500 having a central lumen 504, an exterior tubular wall 505 with at least one channel 503 inside the wall for a control cable to pass through, and a luer lock connector 502 on a proximal end of the handle. The central lumen 504 allows a flexible inner tube or catheter to pass through for delivering a therapeutic agent to a brush. The luer lock connector 502 may connect to a flexible external tube or catheter to allow the therapeutic agent to flow from the external tube or catheter, through the handle and onto a brush. The handle 500 comprises a distal end flexibly connected to an articulating arm 509 made of medical grade catheter material. The articulating arm 509 comprises a central lumen 508 and articulating arm channels 507 for control cables to pass through. The brush head 510 is affixed to a distal end of the articulating arm 509 and can be deflected at various angles controlled by a brush deflector control 501 mounted on the surface of the exterior wall 505 through a slide mechanism. The brush deflector control 501 controls the brush head 510 through control cables passing through the channel 503 inside the exterior tubular wall 505 of the handle. The brush head 510 comprises an open slit 511 for delivering the therapeutic agent onto a brush. The brush head 510 comprises a plurality of strands or sheets of a biocompatible or bioabsorbable material forming a brush. The brush may be of various configurations as shown in FIGS. 8a-8f. A therapeutic agent is delivered through the central lumen 504 which runs through the handle 500, through the deflectable articulating arm 509, and through the brush head 510, and out of the open slit 511 onto the brush.

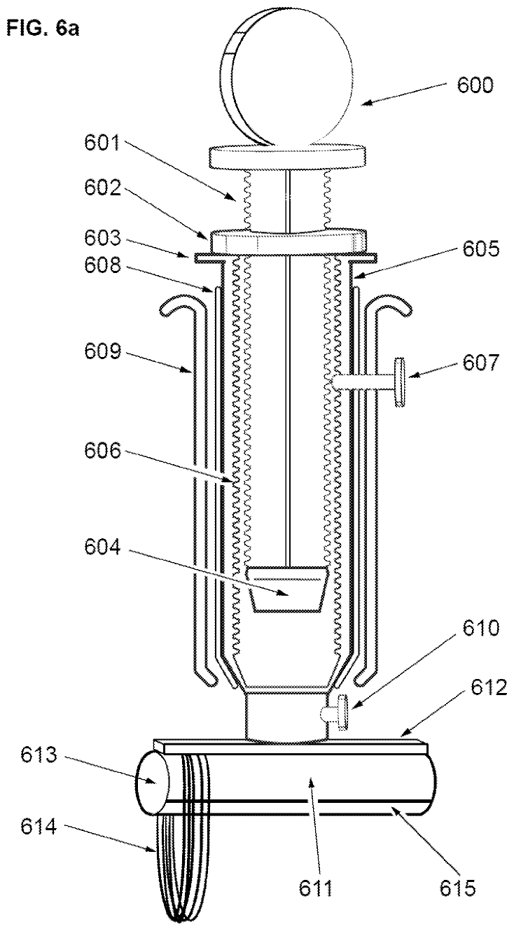

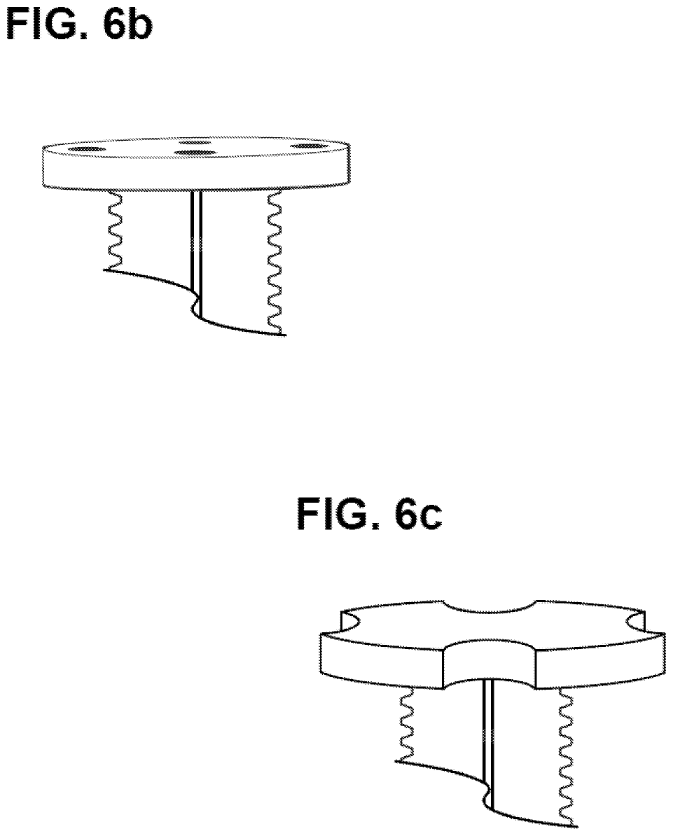

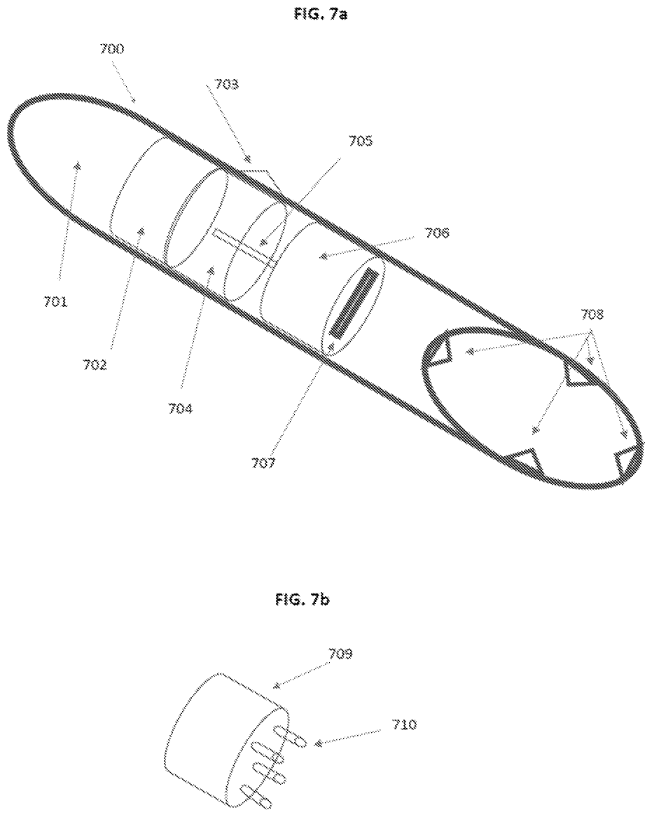

FIG. 6a show a syringe-type device for use in delivery of therapeutic agent(s) according to a sixth embodiment of the invention. The syringe-type device comprises a plunger handle 600 with a disk-like or coin-like member in a vertical orientation permanently affixed on top of another disk-like or coin-like base member in a horizontal orientation. The vertical member is configured to engage a handheld power driver by being inserted into an engagement slit of the syringe power driver. Further details of an engagement slit of the syringe power driver are depicted in FIG. 7a. The plunger handle 600 is mounted on a proximal end of a threaded plunger shaft 601 and may effect a clockwise or counter clockwise rotation of the plunger shaft 601 manually or by a power driver. The threaded plunger shaft 601 has a distal end affixed to a plunger head 604. The threaded plunger shaft 601 includes external threads, which engage internal threads 606 of a syringe barrel 605. Thus, rotating the plunger handle 600 in a clockwise or counter clockwise direction will cause the plunger shaft 601 and plunger head 604 to be moved forward or backward with respect to the syringe barrel 605. The syringe barrel 605 comprises a threaded female shaft access 602 and an upper edge 603 in a proximal end for locking a power driver onto the syringe barrel 605. The syringe barrel 605 further comprises a side port 607 located on the side of the syringe barrel with a stopcock connection or a self-sealing valve for introduction of additional solution or therapeutic agent(s). The syringe barrel 605 may be constructed with a material able to withstand cold temperatures. An insulating layer 608 is formed by coating an insulating material on an external surface of the syringe barrel 605 to maintain the therapeutic temperature. Alternatively, the insulating layer may be formed by coating an insulating material on an internal surface of the syringe barrel 605. A removable insulating sleeve 609 encompassing the syringe barrel 605 provides temperature stabilization for the therapeutic agent(s) or comfortable handling. A one-way stopcock 610 is connected to a distal end of the syringe barrel 605 for air ventilation when an additional material is introduced through the side port 607. The one-way stopcock 610 having a distal end connected to a tubular brush head 611. The one-way stopcock 610 is closed when mixing material within the syringe barrel 605, and is opened when introducing material/solution into the brush head and onto the brush. The brush head 611 comprises an external bar 612, a longitudinal open slit 615, and closed ends 613. The external bar 612 is configured to affix a brush 614 to the brush head 611. The longitudinal open slit 615 in the distal brush head is configured for solution administration onto the brush 614. The brush 614 comprises a plurality of strands or sheets of a biocompatible or bioabsorbable material. The brush may be of various configurations as shown in FIGS. 8a-8f. The strands or sheets could be affixed to the brush head using the external bar 612 with each strand or sheet being tied to the bar, glued to the bar, or crimped between the bar and the brush head. The strands may be looping strands attached to the brush head in a continuous or interrupted fashion. The strands may be grooved to improve retention of the therapeutic agent(s) on the strands. Each strand could either be monofilament, braided, with or without surface grooving to enhance adherence of the therapeutic agent(s). Each strand may be dyed in color or natural. One part of the therapeutic agent(s), such as biologics, may be pre-loaded in the syringe.

Any type of the plunger handle 600 may be employed as should be apparent to those skilled in the art for engagement of a power driver or manual advancement. The plunger handle 600 as shown in FIG. 6a is one illustrative embodiment. In another contemplated embodiment, as shown in FIG. 6b, the plunger handle comprises a flat disk-like or coin-like member having a plurality of holes at the top surface of the member. The number, dimension, and shape of the holes correspond to each respective measurement of the handle engagement pins of a syringe power driver so that the engagement pins can fit into the holes of the plunger handle. Further details of the handle engagement pins of a syringe power driver are depicted in FIG. 7b. Yet in another contemplated embodiment, as shown in FIG. 6c, the plunger handle comprises a flat disk-like or coin-like member optionally having ridges at the edge of the handle for manual advancement.

FIG. 7a show a syringe power driver configured to engage the plunger handle of the syringe-type device as disclosed in FIG. 6a. A syringe power driver for use in a sterile environment or non-sterile environment where administration or delivery of fluids, solutions, or other high viscosity liquids or solutions directly or indirectly to various members within the human body or on the surface of the body is required due to high viscosity of the solution/therapy to be delivered. The syringe power driver comprises an exterior case 700 having a proximal end where a battery/power source 701 is situated; a circuitry 702 situated next to the distal end of the battery/power source 701 to control the revolution speed of the plunger shaft, shaft movement direction (forward or reverse); a switch 703 located on the outside surface of the exterior case 700 with on/off, variable speed, and forward/reverse controls of the rotation of the plunger shaft; a motor 704 situated next to the distal end of the circuitry 702 to drive a drive shaft 705 and a drive shaft extension 706 forward or backward within the exterior case 700; a drive shaft 705 situated in between the motor 704 and a drive shaft extension 706 and having proximal and distal ends connected to the motor and the drive shaft extension, respectively; a drive shaft extension 706 connected to a distal end of the drive shaft 705; an engagement slit 707 in the distal end of the drive shaft extension to engage the syringe plunger handle; and connector ridges 708 at a distal opening of the exterior case 700 to secure the syringe power driver onto the syringe body by twist connection onto the upper edge 602 of the syringe body. It will be appreciated that the configuration of the components and members situated within the exterior case 700 may be varied to obtain desired operations without departing from the scope of the present invention. The exterior case 700 containing all of the above components and members is capable of being used in a sterile or non-sterile surgical environment. The drive shaft 705 along with the drive shaft extension 706 is equal in length to the fully retracted syringe plunger shaft 601 of the syringe-type device as shown in FIG. 6a.

The drive shaft extension 706 may employ any type of the engagement mean as should be apparent to those skilled in the art. In one embodiment, as shown in FIG. 7a, the drive shaft extension 706 comprises an elongated engagement slit 707 which is configured to engage a plunge handle 600 of the device as shown in FIG. 6a by allowing the vertical member of the plunger handle 600 to be inserted into the slit. FIG. 7b depicts another embodiment of the drive shaft extension. The drive shaft extension 709 comprises a plurality of engagement pins 710 which are configured to fit into the holes of the syringe plunger handle as shown in FIG. 6b.

FIGS. 8a-8f present various brush configurations of biocompatible or bioabsorbable material. FIG. 8a shows a brush comprising a plurality of individual looping strands of a biocompatible or bioabsorbable material mounted on the brush head mount in either a “running” fashion or “interrupted” fashion. FIG. 8b shows a brush comprising a plurality of individual single strands of a biocompatible or bioabsorbable material mounted on the brush head mount. FIG. 8c shows a brush comprising a plurality of sheets of a biocompatible or bioabsorbable material stacked or wafered in equal lengths or varying lengths and mounted on the brush head mount. Each sheet may have perforations to improve solution retention on the brush. FIG. 8d shows a brush comprising a single perforated sheet of a biocompatible or bioabsorbable material mounted on the brush head mount. FIG. 8e shows a brush comprising a plurality of solid sheets of a biocompatible or bioabsorbable material stacked or wafered in equal lengths or varying lengths and mounted on the brush head mount. FIG. 8f shows a brush comprising a single solid sheet of a biocompatible or bioabsorbable material mounted on the brush head mount.

FIG. 9 presents a specialized configuration of strands of a biocompatible or bioabsorbable material whereby grooves are created circumferentially in a strand to aid in retention of the therapeutic agent(s) being applied to the organ or tissue of the body. Circumferential grooves 900 are evenly spaced at an interval along each strand to facilitate retention of solution on the entire brush. The strand is made of bioabsorbable suture material 901. The material may be previously FDA approved.

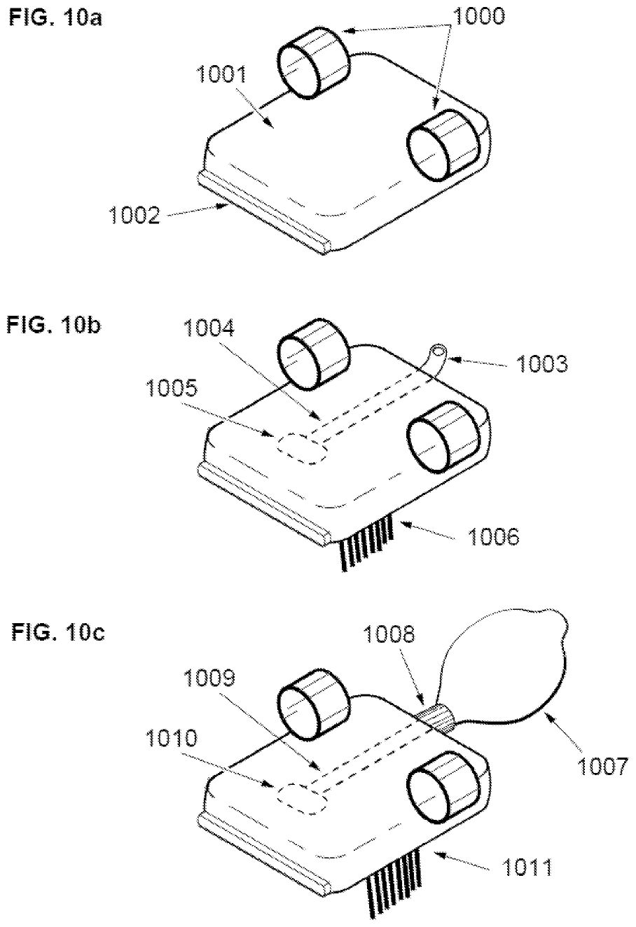

FIGS. 10a-10c depict several embodiments of a device for use in delivery of the therapeutic agent(s) which includes a brush handle with finger controls. As shown in FIG. 10a, the device comprises a platform handle 1000 made of flexible solid medical grade plastic with two finger cylinders 1001 mounted on the platform handle 1000 for allowing to reach around or to the back of the target organ. A brush of various configurations as shown in FIGS. 8a-8f may be affixed to a brush head mount 1002 in a distal end of the platform handle 1000 for delivering the therapeutic agent(s) onto the opposite or posterior surface of the targeted organ. FIG. 10b illustrates another embodiment of the platform handle comprising a proximal port 1003 connected to a channel or tube 1004 within the platform handle terminating into an opening 1005 on the brush side of the platform handle and on the opposite side from the finger controls. The proximal port 1003 of the platform handle may have a luer lock or similar connector to facilitate connection of a tube or syringe to deliver the therapeutic agent(s) or solution through the channel and onto the brush. The brush 1006 may be of a biocompatible or bioabsorbable material in various configurations such as presented in FIGS. 8a-8f. FIG. 10c illustrate another embodiment of the platform handle comprising a flexible or squeezable reservoir 1007 connected on the proximal end of the platform handle and connected to the channel or tube 1009 within the platform handle and terminating into an opening 1010 on the brush side of the platform handle and on the opposite side from the finger controls. The reservoir would have a port or luer lock 1008 for filling the reservoir with a therapy or solution and for connecting with the proximal port of the platform handle. The brush 1011 would be of a biocompatible or bioabsorbable material in various configurations such as presented in FIGS. 8a-8f.

The above description is meant to be exemplary only, and those skilled in the art will recognize that changes may be made to the embodiments without departing from the scope of the present invention. Variations and equivalents to one or more aspects of the invention may employed without departing from the teachings of the present disclosure. Moreover, the present disclosure may be embodied in other specific forms without departing from the subject matter of the claims. Modifications, variations, and equivalents that fall within the scope of the present invention, as should be apparent to those skilled in the art, are intended to fall within the scope of the claims. Also, the scope of the claims is not intended to be limited by the embodiments set forth herein. Instead, the scope of the claims is intended to be given the broadest interpretation consistent with the description as a whole.

### EXAMPLES

It should be understood that the examples and embodiments described herein are for illustrative purposes only and that various modifications or changes in light thereof will be suggested to persons skilled in the art and are to be included within the spirit and purview of this application and the scope of the appended claims. All publications and patent applications mentioned in this specification are indicative of the level of skill of those skilled in the art to which this invention pertains. All publications and patent applications are herein incorporated by reference to the same extent as if each individual publication or patent application was specifically and individually indicated to be incorporated by reference.

(1) Treatment of conduction system disease by gene therapy. The brush could be placed so that the tip portion is in contact with the sinus node at the anterior aspect of the right atrium adjacent to where it connects with the superior vena cava. If desired, electrophysiologic mapping may be used (but would not necessarily be required to) to refine delivery to the sinus node by identifying the site of earliest electrical activation of the right atrium during sinus rhythm. The brush could brush on the solution containing gene transfer vectors (adeno-associated virus, lentivirus, retrovirus, plasmids or other gene transfer vectors) encoding genes known to create automaticity in cardiac myocytes, fibroblast, or other cardiac cells. Examples of these therapeutic genes include HCN1, HCN2, HCN3, HCN4, KCNJ2-AAA, TBX18, adenylate cyclase, SCN4a, ADCY1, and among others. In addition to delivery targeted specifically to the sinus node, the catheter could target any portion of the atria or ventricles where cardiac pacemaker activity might be desired.

(2) Treatment of conduction system disease by gene therapy. In patients with ventricular dyssynchrony, the brush could be used to deliver genes that would induce a Purkinje cell phenotype or an isolated, fast conducting channel (including but not limited to upregulation of GJA1, GJA5, NKX2.5, Tbx 5, Tbx 18, Gata 6, HCN4, KCNE1, KCNE2, SCN1a, SCN10a, SCN5a, Cav3.1, Cav1.3 or Kir3.1, or downregulation of Tbx3, RYR2, Cav1.2, or NCX1) in lines through, across or around the left ventricle to improve conduction and reduce the dyssynchrony.

(3) Treatment of atrial fibrillation by gene therapy. Epicardial gene painting is a target delivery of gene therapy to the atria. This technique consists of brushing the atria a solution containing the gene transfer vector using a brushing device as disclosed previously. Gene transfer vectors encoding genes shown to have antifibrillatory effect or structural remodeling preventive effect would be brushed on the atrial surface (either a subsection or all accessible atrial surfaces). Possible transgenes could include but are not limited to any or all of KCNH2-G628S (or other repolarization prolonging transgene), GJA1, GJA3, GJA5, CAMK2N2, or shRNA directed against CASP3, TGFβ, TNFα, KCNH2, KCHQ1. The gene transfer vector could be delivered in saline or other liquid medium or in complex with matrigel, poloxamer or other agent to assist in vector retention. The atrial epicardial gene painting is a widespread atrial gene transfer strategy that achieves dense, transmural, homogenous atrial expression of therapeutic gene without affecting ventricular cardiomyocytes. Atrial gene painting of the gene transfer vector encoding HERG-G628S involves applying a mixture of poloxamer gel, dilute trypsin and gene transfer vector to the atrial epicardial surface. See Kikuchi K et al. Targeted modification of atrial electrophysiology by homogeneous transmural atrial gene transfer. Circulation. 2005; 111:264-70. Poloxamer gel is used to increase virus contact time with the atria and trypsin increases virus penetration. In clinical settings, this delivery method could potentially be performed during open cardiac surgery or cardiac allografting. Another atrial gene painting example for the treatment of atrial fibrillation is to deliver viral vectors encoding light-activatable ion channels with an implantable light source and rhythm detector, developing an autogenous arrhythmia termination system. This optogenetic approach restored normal heart rhythm in rat hearts ex vivo and in vivo under closed-chest conditions. See Nyns E. et al. An automated hybrid bioelectronic system for autogenous restoration of sinus rhythm in atrial fibrillation. Science Translational Medicine. 2019: Vol. 11, Issue 481.

(4) Treatment of atrial fibrillation by pharmacotherapy. Antifibrillatory drugs (amiodarone, dofetilide, sotalol, flecainide, propafenone) complexed in retaining agent(s) (pluronics, matrigel, polylysine, collagen, carbohydrate polymers) would be brushed on all exposed atrial surfaces or on strategic parts of the atria from which they could be distributed locally (e.g. delivery between the pulmonary veins in the posterior left atrium, or on the atrial free walls).

(5) Treatment of heart failure by gene therapy. The brush system can apply gene transfer vectors with or without a matrix for retention onto the ventricles. The gene transfer vectors would encode CaMK2N2, SERCA1, SERCA2a, angiogenesis genes (VEGF, FGF, PDGF), antiapoptotic genes (CASP3 inhibitory shRNA), anti-inflammatory genes (TGFβ or TNFα inhibitory shRNA) or corrective genes for dilated or hypertrophic or arrhythmogenic cardiomyopathies, or other genes that have benefit for improvement of left ventricular function.

In one embodiment, the device could be packaged in a kit, which may include a brush to apply the solutions to the desired areas of the organ(s). The gene transfer solutions described herein, e.g., gene transfer vectors, infusion solutions, or complexing matrices could additionally be included in these kits. Accessory components of this embodiment could also be included in the kit.

1.-107. (canceled)

108. A device for delivering at least one therapeutic agent to an organ or tissue for treating a disease, comprising:
a brush of a synthetic absorbable material; and
a handle, wherein a distal end of the handle is affixed to the brush or a brush head.

109. The device of claim 108, the brush comprises a plurality of strands.

110. The device of claim 109, wherein the strands are single or looping strands attached to the handle or the brush head in a continuous or interrupted fashion.

111. The device of claim 109 wherein the strands comprise circumferential grooves for improving retention of the therapeutic agent on the strand.

112. The device of claim 109, wherein the circumferential grooves are evenly spaced at an interval along each strand to facilitate retention of solution.

113. The device of claim 109, wherein each strand is monofilament or braided.

114. The device of claim 108, the brush comprises one sheet or a plurality of sheets.

115. The device of claim 114, wherein the plurality of sheets is stacked or wafered in equal lengths or varying lengths.

116. The device of claim 114, wherein the sheet has perforations configured to improve solution retention on the brush.

117. The device of claim 114, wherein the sheet is a solid sheet without perforations.

118. The device of claim 108, wherein the synthetic absorbable material comprises polydioxanone (PDS), polylactic acid (PLA), polyglycolic acid (PGA), Polycaprolactone (PCL), and polylactic-co-glycolic acid (PLGA).

119. The device of claim 108, wherein the handle is a straight handle or a canted handle.

120. The device of claim 108, wherein the handle is a plunger handle of the syringe-type.

121. The device of claim 120, wherein the plunger handle comprises a syringe power driver configured to drive a plunger shaft of the device.

122. The device of claim 108, wherein the handle is a platform handle having one or more finger controls mounted on a proximal end of the handle and a brush at a distal end of the handle.

123. The device of claim 108, wherein the handle comprises a central lumen for delivering the therapeutic agent and an exterior tubular wall with at least one channel within the wall for a control cable to pass through.

124. The device of claim 108, wherein the brush head comprises an open slit for delivery of the therapeutic agent onto the brush and an external bar configured for affixing the brush to the brush head.

125. The device of claim 108, further comprises a brush deflector control mounted on the handle configured to deflect the brush head at various angles.

126. The device of claim 108, wherein the therapeutic agent comprises genetically engineered vectors or cells, proteins, stem cells, small molecule pharmaceuticals, and biologics.

127. The device of claim 108, wherein the disease comprises cardiac diseases selected from the group consisting of conduction system disease, sinus node dysfunction, atrial fibrillation, myocardial infarction, and heart failure.
